(19) United States
(12) Patent Application Publication
(10) Pub. No.: US 2022/0409086 A1
(43) Pub. Date: Dec. 29, 2022

(54) System for Strategic Monitoring and Treatment of Pressure Ulcer Using Sub-Epidermal Moisture Values

(71) Applicant: Bruin Biometrics, LLC, Los Angeles, CA (US)

(72) Inventor: Martin F. BURNS, Los Angeles, CA (US)

(73) Assignee: Bruin Biometrics, LLC, Los Angeles, CA (US)

(21) Appl. No.: 17/860,222

(22) Filed: Jul. 8, 2022

Related U.S. Application Data
(63) Continuation of application No. 16/684,573, filed on Nov. 14, 2019, now abandoned.
(60) Provisional application No. 62/901,667, filed on Sep. 17, 2019.

(51) Int. Cl. A61B 5/0537; A61B 5/00; A61B 5/0531

(52) U.S. Cl. CPC A61B 5/0537; A61B 5/7495; A61B 5/445; A61B 5/0531

(57) ABSTRACT

The present disclosure provides systems and methods for detecting and monitoring a patient in need of pressure ulcer treatment based on measurements of Sub-Epidermal Moisture (SEM) values.

### CROSS-REFERENCE TO RELATED APPLICATIONS

This application is a continuation of U.S. application Ser. No. 16/684,573 filed Nov. 14, 2019, which claims the benefit of U.S. Provisional Application No. 62/901,667 filed Sep. 17, 2019, the entire contents of each of which are incorporated herein by reference.

### FIELD

The present disclosure provides systems and methods for detecting and monitoring a patient in need of pressure ulcer treatment based on measurements of Sub-Epidermal Moisture (SEM) values.

### BACKGROUND

The skin is the largest organ in the human body. It is readily exposed to different kinds of damages and injuries. When the skin and its surrounding tissues are unable to redistribute external pressure and mechanical forces, ulcers may be formed. Prolonged continuous exposure to even modest pressure, such as the pressure created by the body weight of a supine patient on their posterior skin surfaces, may lead to a pressure ulcer.

Pressure ulcers are developed by approximately 2.5 million people a year in the United States and an equivalent number in the European Union. In long-term and critical-care settings, up to 25% of elderly and immobile patients develop pressure ulcers. Approximately 60,000 U.S. patients die per year due to infection and other complications from pressure ulcers.

Detecting tissue damage before the skin breaks and intervening with the appropriate therapy to avoid further deterioration of the underlying tissue is desirable not only for the patient but society. The average cost of treating pressure-induced damage at the earliest visible sign (a Stage 1 ulcer) is only $2,000 but this rises to $129,000 when the ulcer is deep enough to expose muscle or bone (a Stage 4 ulcer). The current standard to detect pressure ulcers is by visual inspection, which is subjective, unreliable, untimely, and lacks specificity.

### SUMMARY

In an aspect, the present disclosure provides for, and includes, a system for monitoring a patient in need of pressure ulcer treatment, the system comprising: a sub-epidermal moisture (SEM) scanner comprising a barcode scanner; a first barcode identifying the patient, a second barcode identifying a caretaker; and a third barcode identifying an intervention being applied to the patient. In an aspect, an SEM scanner further comprises one or more electrodes capable of interrogating target tissue of the patient, a circuit that is electronically coupled to the one or more electrodes to form a sensor, generate a bioimpedance signal, and convert the bioimpedance signal into an SEM value; a processor electronically coupled to the circuit and configured to receive the SEM values; and a non-transitory computer readable media that is electronically coupled to the process and comprises instructions stored thereon that, when executed on the processor, performs the steps of receiving an SEM value measured at the target tissue; receiving the first barcode, receiving the second barcode, and receiving a third barcode. In an aspect, the system further comprises a database capable of receiving and storing the SEM value, and the first, second, and third barcodes collected by the SEM scanner.

In an aspect, the present disclosure provides for, and includes, a method of assigning an intervention to a patient in need of pressure ulcer treatment, the method comprising: receiving a sub-epidermal moisture (SEM) value, a patient barcode, and a current intervention barcode from an SEM scanner in accordance with the present disclosure; transferring the SEM value, the patient barcode, and the current intervention barcode to a database; identifying data stored in the database associated with the patient barcode; comparing the SEM value to existing SEM values stored in the database; assigning a new intervention based on the comparing step.

In an aspect, the present disclosure provides for, and includes, a method of tracking compliance of patient receiving a pressure ulcer treatment, the method comprising: receiving a sub-epidermal moisture (SEM) value, a patient barcode, and a current intervention barcode from an SEM scanner in accordance with the present disclosure; transferring the SEM value, the patient barcode, and the current intervention barcode to a database; identifying data stored in the database associated with the patient barcode; comparing the current intervention to the intervention stored in the database.

In an aspect, the present disclosure provides for, and includes, a method of tracking intervention assets in an institution, the method comprising receiving a sub-epidermal moisture (SEM) value, a patient barcode, a caretaker barcode, and a current intervention barcode from an SEM scanner in accordance with the present disclosure; transferring and storing the SEM value, the patient barcode, the caretaker barcode, and the current intervention barcode to a database; identifying data stored in the database associated with the patient barcode; receiving an inquiry intervention asset barcode; identifying the patient associated with the barcode; locating the inquiry intervention asset.

### BRIEF DESCRIPTION OF THE DRAWINGS

Aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIG. 1A and 1B depict an SEM scanner comprising a barcode scanner, in accordance with the present description.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Thus, the disclosure contemplates that, in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular embodiments of the disclosure, and not to exhaustively specify all permutations, combinations, and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure.

All publications, patent applications, patents, and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

U.S. patent application Ser. No. 14/827,375 (“the '375 application”) discloses an apparatus that measures the sub-epidermal capacitance using a bipolar sensor, where the sub-epidermal capacitance corresponds to the moisture content of the target region of skin of a patient. The '375 application also discloses an array of these bipolar sensors of various sizes.

U.S. patent application Ser. No. 15/134,110 discloses an apparatus for measuring sub-epidermal moisture (SEM), where the device emits and receives an RF signal at a frequency of 32 kHz through a single coaxial sensor and generates a bioimpedance signal, then converts this signal to an SEM value.

U.S. patent application Ser. Nos. 15/887,831, 15/887,883, 15/887,886, and 15/887,837 disclose additional apparatuses for measuring SEM values.

U.S. patent application Ser. Nos. 16/193,636 and 16/193,707 disclose workflow strategies for identifying, monitoring, and treating patients in need of pressure ulcer treatments using SEM values.

U.S. patent application Ser. No. 13/942,649 discloses a compact perfusion scanner and method of characterizing tissue heath status incorporating optical sensors to monitor tissue blood perfusion measurements and oximetry.

U.S. patent application Ser. No. 16/511,802 discloses workflow strategies for identifying, monitoring, and treating patients in need of pressure ulcer treatments using tissue blood perfusion measurements and oximetry.

The aforementioned U.S. patent application Ser. Nos. 14/827,375, 15/134,110, 15/887,831, 15/887,883, 15/887,886, 15/887,837, 16/193,636, 16/193,707, 13/942,649 and 16/511,802 are incorporated herein by reference in their entireties.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein include and comprise one or more steps or actions for achieving the described method. The method steps and/or actions may be interchanged with one another without departing from the scope of the present disclosure. In other words, unless a specific order of steps or actions is required for proper operation of the embodiment, the order and/or use of specific steps and/or actions may be modified without departing from the scope of the present disclosure.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or an SEM value and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “sub-epidermal moisture” or “SEM” refers to the increase in tissue fluid and local edema caused by vascular leakiness and other changes that modify the underlying structure of the damaged tissue in the presence of continued pressure on tissue, apoptosis, necrosis, and the inflammatory process.

As used herein, a “patient” may be a human or animal subject.

As used herein, “delta” refers to a calculated difference between two SEM values.

The present disclosure provides systems and methods for detecting and monitoring a patient in need of pressure ulcer treatment based on measurements of Sub-Epidermal Moisture (SEM) value.

In an aspect, the present disclosure provides for, and includes, a system for monitoring a patient in need of pressure ulcer treatment, the system comprising: a sub-epidermal moisture (SEM) scanner comprising a barcode scanner; a first barcode identifying the patient, a second barcode identifying a caretaker; and a third barcode identifying an intervention being applied to the patient. In an aspect, an SEM scanner further comprises one or more electrodes capable of interrogating target tissue of the patient, a circuit that is electronically coupled to the one or more electrodes to form a sensor, generate a bioimpedance signal, and convert the bioimpedance signal into an SEM value; a processor electronically coupled to the circuit and configured to receive the SEM values; and a non-transitory computer readable media that is electronically coupled to the process and comprises instructions stored thereon that, when executed on the processor, performs the steps of receiving an SEM value measured at the target tissue; receiving the first barcode, receiving the second barcode, and receiving a third barcode. In an aspect, the system further comprises a database capable of receiving and storing the SEM value, and the first, second, and third barcodes collected by the SEM scanner.

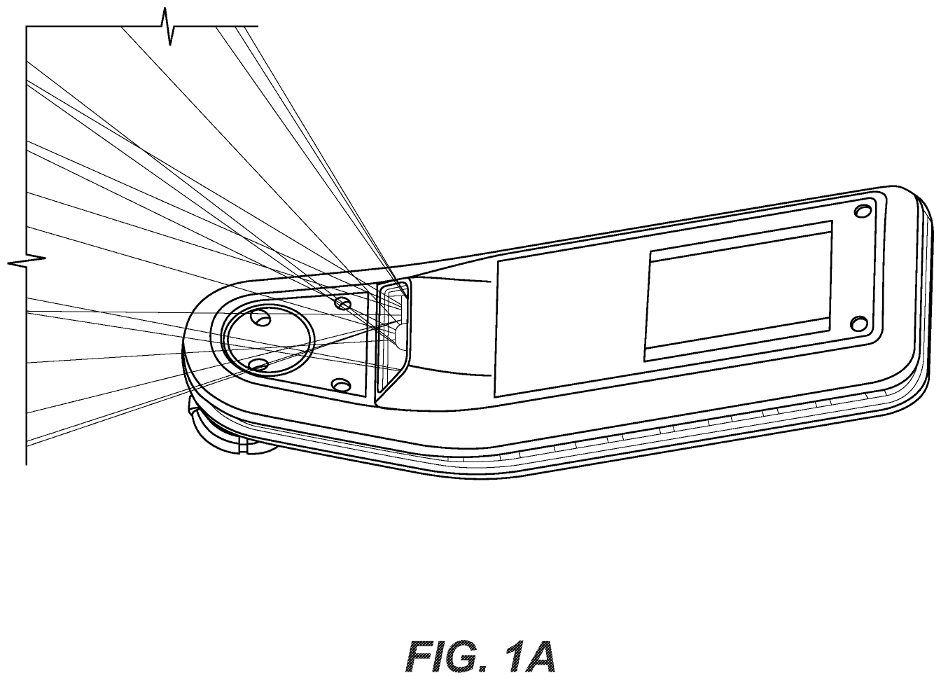

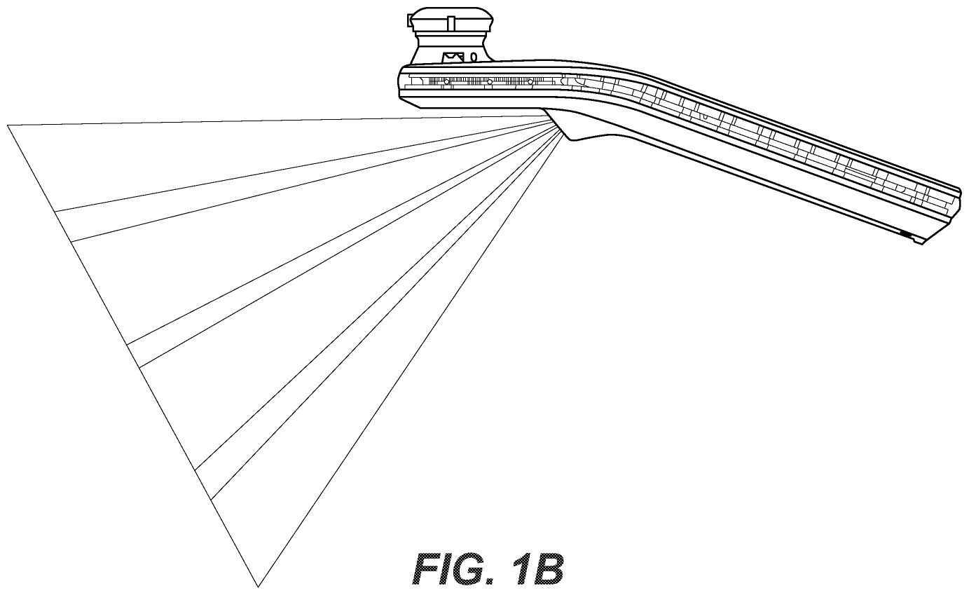

FIGS. 1A and 1B depict an example SEM scanner of the system in accordance with the present disclosure. In particular, FIGS. 1A and 1B depict an SEM scanner having a built-in barcode scanner on its back side. In an aspect, a barcode scanner can be a laser scanner. In an aspect, a barcode scanner can be a camera. In an aspect, a barcode scanner detects a 1D barcode. In an aspect, a barcode scanner detects a 2D barcode in the form of a QR code. In an aspect, a barcode of a system of the present disclosure can be replaced with a unique identifier. In an aspect, a system comprises a keypad for manual entry of a unique identifier. In an aspect, the system in accordance with the present disclosure may be used with a device detecting a measurement selected from the group consisting of bioimpedance, biocapacitance, oximetry, tissue perfusion, ultrasound, pressure measurement; capillary pressure, thermal imaging, spectral imaging, transcutaneous water loss, and detection of interleukin-1 alpha presence at one or more anatomic site of interest.

In an aspect, an SEM scanner in accordance with the present disclosure comprises a sensor formed from a plurality of electrodes such as up to two electrodes, up to three electrodes, up to four electrodes, up to five electrodes, up to six electrodes, up to seven electrodes, up to eight electrodes, up to nine electrodes, up to ten electrodes, up to eleven electrodes, or up to twelve electrodes. In an aspect, an SEM scanner comprises a plurality of sensors formed from a plurality of electrodes, where each sensor is formed from up to twelve electrodes, such as up to two electrodes, up to three electrodes, up to four electrodes, up to five electrodes, up to six electrodes, up to seven electrodes, up to eight electrodes, up to nine electrodes, up to ten electrodes, or up to eleven electrodes. In an aspect, a sensor is formed from an annular ring disposed around an inner circular electrode. In an aspect, a sensor is formed from two parallel bar electrodes. In an aspect, a sensor is formed from electrodes in the form of interdigitating fingers. In an aspect, an SEM scanner further comprises a plurality of contact sensors selected from the group consisting of a plurality of bioimpedance sensors, a plurality of pressure sensors, a plurality of light sensors, a plurality of temperature sensors, a plurality of pH sensors, a plurality of perspiration sensors, a plurality of ultrasonic sensors, a plurality of bone growth stimulator sensors, or a plurality of a combination of these sensors. In an aspect, an SEM scanner further comprises a plurality of light sensors. In an aspect, an SEM scanner further comprises one or more light emitting sources comprising dual emitters configured for emitting 660 nm and 880 nm light. In an aspect, an SEM scanner further comprises an insulating cover layer on top of its electrodes, forming a barrier between the electrodes and the patient's skin while measurements are being taken.

In an aspect, an SEM scanner of the system in accordance with the present disclosure can be an SEM scanner described in any one of U.S. patent application Ser. Nos. 14/827,375, 15/134,110, 15/887,831, 15/887,883, 15/887,886, 15/887,837, which are incorporated herein by reference in their entireties.

In an aspect, a first barcode identifying a patient is printed as a part of a bracelet to be worn by the patient. In an aspect, a first barcode identifying a patient is printed on a sheet of paper to be placed alongside the patient's bed.

In an aspect, a second barcode identifying a caretaker is printed as a part of a an employee identification card to be worn by the caretaker. In an aspect, a caretaker can be a physician, a nurse practitioner, a nurse, a physician assistant, or a healthcare worker.

In an aspect, a third barcode identifying an intervention asset is printed as a label and is affixed to the intervention asset. In an aspect, a third barcode identifying an intervention asset is printed as a label and is affixed to the packaging of an intervention asset. In an aspect, an intervention asset is selected from the group consisting of a specialized support surface, a heel boot, a dressing, a low-friction sheet cover, a low-friction padded mattress surface, a wedge, a container of barrier cream, a container of topical cream for enhancing perfusion, an accelerometer measuring patient movement, and a silicone pad.

In an aspect, a database is a secured cloud-based database for storing data indexed by patient identification or caretaker identification.

In an aspect, the present disclosure provides for, and includes, a method of assigning an intervention to a patient in need of pressure ulcer treatment, the method comprising: receiving a sub-epidermal moisture (SEM) value, a patient barcode, and a current intervention barcode from an SEM scanner in accordance with the present disclosure; transferring the SEM value, the patient barcode, and the current intervention barcode to a database; identifying data stored in the database associated with the patient barcode; comparing the SEM value to existing SEM values stored in the database; assigning a new intervention based on the comparing step.

In an aspect, an SEM scanner transfers data to a database via Wi-Fi or Bluetooth connection.

In an aspect, the step of comparing and assigning a new invention are performed in accordance with that described in U.S. patent application Ser. Nos. 16/193,636, 16/193,707, and 16/511,802, which are incorporated herein by reference in their entireties.

In an aspect, the present disclosure provides for, and includes, a method of tracking compliance of patient receiving a pressure ulcer treatment, the method comprising: receiving a sub-epidermal moisture (SEM) value, a patient barcode, and a current intervention barcode from an SEM scanner in accordance with the present disclosure; transferring the SEM value, the patient barcode, and the current intervention barcode to a database; identifying data stored in the database associated with the patient barcode; comparing the current intervention to the intervention stored in the database.

In an aspect, the present disclosure provides for, and includes, a method of tracking intervention assets in an institution, the method comprising receiving a sub-epidermal moisture (SEM) value, a patient barcode, a caretaker barcode, and a current intervention barcode from an SEM scanner in accordance with the present disclosure; transferring and storing the SEM value, the patient barcode, the caretaker barcode, and the current intervention barcode to a database; identifying data stored in the database associated with the patient barcode; receiving an inquiry intervention asset barcode; identifying the patient associated with the barcode; locating the inquiry intervention asset.

In an aspect, an institution is selected from the group consisting of a hospital, a long-term care facility, and an old age home.

While the invention has been described with reference to particular embodiments, it will be understood by those skilled in the art that various changes may be made and equivalents may be substituted for elements thereof without departing from the scope of the invention. In addition, many modifications may be made to a particular situation or material to the teachings of the invention without departing from the scope of the invention. Therefore, it is intended that the invention not be limited to the particular embodiments disclosed but that the invention will include all embodiments falling within the scope and spirit of the appended claims.

1. A system for monitoring a patient in need of pressure ulcer treatment, the system comprising:
a sub-epidermal moisture (SEM) scanner comprising a barcode scanner;
a first barcode identifying the patient,
a second barcode identifying a caretaker;
and a third barcode identifying an intervention being applied to the patient.

2. The system of claim 1, wherein the SEM scanner further comprises:
one or more electrodes capable of interrogating target tissue of the patient,
a circuit that is electronically coupled to the one or more electrodes to form a sensor, generate a bioimpedance signal, and convert the bioimpedance signal into an SEM value;
a processor electronically coupled to the circuit and configured to receive the SEM values; and
a non-transitory computer readable media that is electronically coupled to the process and comprises instructions stored thereon that, when executed on the processor, performs the steps of receiving an SEM value measured at the target tissue, receiving the first barcode, receiving the second barcode, and receiving a third barcode.

3. The system of claim 1, further comprising a database capable of receiving and storing the SEM value, and the first, second, and third barcodes collected by the SEM scanner.

4. A method of assigning an intervention to a patient in need of pressure ulcer treatment, the method comprising:
receiving a sub-epidermal moisture (SEM) value, a patient barcode, and a current intervention barcode from an SEM scanner;
transferring the SEM value, the patient barcode, and the current intervention barcode to a database;
identifying data stored in the database associated with the patient barcode;
comparing the SEM value to existing SEM values stored in the database; and
assigning a new intervention based on the comparing step.

5. A method of tracking compliance of patient receiving a pressure ulcer treatment, the method comprising:
receiving a sub-epidermal moisture (SEM) value, a patient barcode, and a current intervention barcode from an SEM scanner;
transferring the SEM value, the patient barcode, and the current intervention barcode to a database;
identifying data stored in the database associated with the patient barcode; and
comparing the current intervention to the intervention stored in the database.

6. A method of tracking intervention assets in an institution, the method comprising:
receiving a sub-epidermal moisture (SEM) value, a patient barcode, a caretaker barcode, and a current intervention barcode from an SEM scanner;
transferring and storing the SEM value, the patient barcode, the caretaker barcode, and the current intervention barcode to a database;
identifying data stored in the database associated with the patient barcode;
receiving an inquiry intervention asset barcode;
identifying the patient associated with the barcode; and
locating the inquiry intervention asset.

7. The system of claim 2, wherein the sensor is formed from an annular ring disposed around an inner circular electrode.

8. The system of claim 1, wherein the first barcode identifying the patient is printed as a part of a bracelet to be worn by the patient.

9. The system of claim 1, wherein the first barcode identifying the patient is printed on a sheet of paper to be placed alongside the patient's bed.

10. The system of claim 1, wherein the second barcode identifying the caretaker is printed as part of an employee identification card to be worn by the caretaker.

11. The system of claim 1, wherein the caretaker is selected from the group consisting of a physician, a nurse practitioner, a nurse, a physician assistant, and a healthcare worker.

12. The system of claim 1, wherein the third barcode identifying the intervention being applied to the patient is printed as a label and is affixed to the intervention asset.

13. The system of claim 1, wherein the third barcode identifying the intervention being applied to the patient is printed as a label and is affixed to the packaging of the intervention asset.

14. The system of claim 12, wherein the intervention asset is selected from the group consisting of a specialized support surface, a heel boot, a dressing, a low-friction sheet cover, a low-friction padded mattress surface, a wedge, a container of barrier cream, a container of topical cream for enhancing perfusion, an accelerometer measuring patient movement, and a silicone pad.

15. The system of claim 13, wherein the intervention asset is selected from the group consisting of a specialized support surface, a heel boot, a dressing, a low-friction sheet cover, a low-friction padded mattress surface, a wedge, a container of barrier cream, a container of topical cream for enhancing perfusion, an accelerometer measuring patient movement, and a silicone pad.

16. The system of claim 3, wherein the database is a secured cloud-based database for storing data indexed by patient identification or caretaker identification.
